(19) United States
(12) Patent Application Publication
(10) Pub. No.: US 2026/0061219 A1
(43) Pub. Date: Mar. 5, 2026

(54) Far-UVC Treatment

(71) Applicant: Sensor Electronic Technology, Inc., Columbia, SC (US)

(72) Inventors: Piyush Sharma, Columbia, SC (US); Carlton James Gibson, Columbia, SC (US)

(21) Appl. No.: 19/305,127

(22) Filed: Aug. 20, 2025

Related U.S. Application Data
(60) Provisional application No. 63/689,101, filed on Aug. 30, 2024.

(51) Int. Cl. A61N 5/06

(52) U.S. Cl. CPC A61N 5/0624; A61N 2005/0627; A61N 2005/0632; A61N 2005/0636; A61N 2005/0661

(57) ABSTRACT

A surface treatment system can include a reconfigurable frame structure at least partially defining a treatment area. The frame structure can include panels configured for repeated assembly and disassembly in any of a plurality of configurations and far-UVC light sources configured to emit far-UVC light directed into the treatment area. The surface treatment system also can include sensors configured to acquire presence data corresponding to a presence of an object in the treatment area and a control unit configured to operate the plurality of far-UVC light sources to treat surfaces of the object located in the treatment area using the presence data.

### REFERENCE TO RELATED APPLICATIONS

The current application claims the benefit of U.S. Provisional Application No. 63/689,101, filed on 30 Aug. 2024, which is hereby incorporated by reference.

### TECHNICAL FIELD

The disclosure relates generally to ultraviolet-based treatment, and more particularly, to ultraviolet treatment using far-UVC radiation.

### BACKGROUND ART

The prevailing landscape for disinfection methodologies predominantly revolves around established techniques such as chemical sanitization, UV-C irradiation, and thermal treatments. While these approaches have been effective in mitigating the spread of pathogens, they exhibit inherent limitations that hinder their efficacy, particularly in environments necessitating rapid and comprehensive disinfection measures.

One notable challenge within the current state of the art lies in the utilization of UV-C irradiation for disinfection purposes. While UV-C light is effective in neutralizing a broad spectrum of microorganisms, its application is impeded by concerns regarding human safety. Prolonged exposure to UV-C radiation can lead to adverse effects on human skin, eyes, and hair, thereby constraining its practicality in scenarios where direct contact with the UV-C radiation is inevitable or even possible.

Existing approaches for providing disinfection to high human foot traffic areas must circumvent the limitations of conventional UV-C irradiation, e.g., by implementing protective barriers or shields to mitigate direct human exposure to UV light. However, such approaches introduce practical challenges and drawbacks, including compromised disinfection efficiency due to obstruction of UV light, heightened maintenance demands for cleaning and replacing barriers, and potential user inconvenience.

### SUMMARY OF THE INVENTION

Aspects of the invention provide a solution for treating a surface, such as human skin and/or clothing being worn by a human, as described herein.

Embodiments of the invention can integrate far-UVC technology into disinfection mechanisms. Far-UVC light operates within the 200-225 nm, and has unique characteristics that distinguish it from the longer wavelength UV-C. Notably, far-UVC light is inherently safer for human exposure, exhibiting negligible adverse effects especially resulting from brief periods of contact.

By leveraging far-UVC technology, embodiments of the invention can provide UV-C-based disinfection in new applications. For example, UV-C disinfection can be used in high-traffic settings, while ensuring both efficacy and safety. More particular embodiments can be directed to areas in which humans are moving. In such embodiments, a door frame or a cylindrical walk-in structure can comprise far-UVC light sources positioned to facilitate comprehensive disinfection of some or all of the surfaces of people passing therethrough. Such a configuration can provide pathogen eradication while also minimizing any risk of harm to the individuals traversing through the disinfection zone.

Embodiments of the proposed invention can offer a holistic and user-friendly disinfection solution grounded in far-UVC technology, which can reduce or eliminate the need for protective barriers. Embodiments of the invention can streamline the disinfection process while safeguarding human well-being.

A surface treatment system described herein can include a reconfigurable frame structure at least partially defining a treatment area. The frame structure can include panels configured for repeated assembly and disassembly in any of a plurality of configurations and far-UVC light sources configured to emit far-UVC light directed into the treatment area. The surface treatment system also can include sensors configured to acquire presence data corresponding to a presence of an object in the treatment area and a control unit configured to operate the plurality of far-UVC light sources to treat surfaces of the object located in the treatment area using the presence data.

A first aspect of the invention provides a surface treatment system comprising: a reconfigurable frame structure defining at least two sides of a treatment area located between the at least two sides, wherein the frame structure comprises a plurality of panels configured for repeated assembly and disassembly in any of a plurality of configurations; a plurality of far-UVC light sources located in the frame structure, wherein the plurality of far-UVC light sources are configured to emit far-UVC light directed into the treatment area; a set of motion sensors configured to acquire presence data corresponding to a presence of an object in the treatment area; and a control unit configured to operate the plurality of far-UVC light sources to treat surfaces of the object located in the treatment area using the presence data.

A second aspect of the invention provides a treatment system comprising: a frame structure comprising a plurality of panels defining a top and two sides of a treatment area, wherein each of the plurality of panels includes at least one far-UVC light source configured to emit far-UVC light directed into the treatment area; a set of sensors configured to acquire presence data corresponding to a presence of an object in the treatment area; and a control unit configured to operate the plurality of far-UVC light sources to treat surfaces of the object using the presence data.

A third aspect of the invention provides a method of treating surfaces associated with a human, the method comprising: assembling a reconfigurable frame structure including a plurality of panels configured for repeated assembly and disassembly into one of a plurality of configurations, wherein the assembled reconfigurable frame structure at least partially defines a treatment area, and wherein each of the plurality of panels includes at least one far-UVC light source configured to emit far-UVC light directed into the treatment area; acquiring presence data corresponding to a presence of the human in the treatment area using a set of motion sensors; and a control unit operating the plurality of far-UVC light sources to treat the surfaces associated with the human located in the treatment area using the presence data, wherein the surfaces include human skin and clothing being worn by a human.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative ultraviolet treatment system according to embodiments.

FIG. 2 shows an illustrative configuration of a treatment system located in front of a door according to embodiments.

FIG. 3 shows an illustrative treatment system according to embodiments.

FIG. 4 shows an illustrative treatment system according to embodiments.

FIGS. 5A and 5B show schematic front and right side views, respectively, of an illustrative connection interface between two panels according to embodiments, and

FIG. 5C shows a schematic of an illustrative corner connector according to embodiments.

FIG. 6 shows an illustrative schematic diagram of operation of a treatment system according to embodiments.

FIG. 7 shows a schematic of an illustrative treatment system according to embodiments.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As discussed herein, embodiments provide a treatment system for treating a surface with far-UVC light. More particular embodiments can be directed to treating human skin and/or clothing being worn by a human with far-UVC light.

As used herein, “treatment” includes directing germicidal radiation onto one or more surfaces. In embodiments, the surface(s) can include surfaces associated with a human, including human skin, clothing, and/or the like. The treatment can result in one or more contaminants (e.g., pathogens) present on the surface(s) to be killed, inactivated, and/or the like. Embodiments of the treatment can include a dose sufficient to decontaminate, disinfect, sterilize, and/or the like, a treated surface to a desired level. It is understood that embodiments of the treatment described herein may not result in a surface being fully decontaminated, disinfected, sterilized, and/or the like. While susceptibility to UV light varies, exposure to UV energy for about 20 to about 34 milliwatt-seconds/cm² is adequate to deactivate approximately 99 percent of the pathogens.

Embodiments utilize properties of far-UVC light, a specific wavelength range within ultraviolet light that is inherently safer for human exposure compared to conventional UVC radiation. As used herein, ultraviolet (UV) light includes radiation having a wavelength between 10 nanometers and 400 nanometers. Ultraviolet C (UVC) light is a subtype of ultraviolet light having wavelengths between 100 nanometers and 280 nanometers. Far-UVC light is a subtype of UVC light having wavelengths between 200 nanometers and 235 nanometers. In more particular embodiments, far-UVC light having wavelengths between 207 nanometers and 222 nanometers can be utilized.

Embodiments of a far-UVC treatment system described herein can be configured for various environments and spatial constraints. For example, embodiments can be configured for use as an entry frame, e.g., for a door or other entrance, a walk-in booth, and/or the like. Entry frame embodiments can provide a compact yet effective solution, which can be particularly suitable for installations where space is limited, a discreet aesthetic is desired, a comprehensive treatment is not required, and/or the like. In the entry frame configuration, one or more far-UVC light sources can be integrated into a top and/or sides of a frame structure located such that individuals pass therethrough when entering or exiting an area. Conversely, walk-in booth embodiments can offer comprehensive disinfection coverage, which can help ensure thorough sanitation while minimizing any limitations associated with confined spaces. The walk-in booth configuration can comprise far-UVC light sources positioned on the bottom, top, and/or sides of a booth structure. Such a configuration can provide comprehensive treatment coverage for an individual located in and/or passing through the booth.

To mitigate any potential risks associated with far-UVC exposure, embodiments can comprise one or more integrated safety features. For example, embodiments can comprise one or more motion sensors, presence sensors, and/or the like, which can generate signals to automatically deactivate far-UVC light source(s) when no individuals are present within a treatment area. Additionally, embodiments can generate visual and/or audible cues which can provide indications of system activation. Embodiments can comprise a timer, which times a duration of a treatment and can deactivate the far-UVC light source(s) when an individual has received a maximum treatment.

Embodiments can be scalable. To this extent, embodiments can be designed to be adaptable to various doorway or room sizes, which can provide an effective treatment solution for diverse applications. Embodiments can comprise flexible design elements, which can enable easy customization and/or adjustment, provide compatibility with a range of spatial configurations, and/or the like, while providing effective treatment performance.

Embodiments can comprise one or more far-UVC light sources, one or more sensors, one or more visual and/or audible indicators, and a control unit, which can comprise one or more computing devices.

Each far-UVC light source can emit ultraviolet light having wavelengths within the far-UVC wavelength range, which is generally considered safe for humans at sufficiently low doses and can effectively neutralize pathogens, thereby providing effective treatment while posing minimal risk to human health. In embodiments, one or more far-UVC light emitting diodes (LEDs) are used to generate the far-UVC light.

A sensor can detect the presence or absence of an individual entering the disinfection zone, which can trigger activation or deactivation of the far-UVC light source(s). Embodiments can utilize one or more of any type of sensor. A non-exhaustive list of sensors includes proximity sensors, such as light and/or motion detectors, which can detect the presence of users within or in the vicinity of a treatment area. Embodiments can include additional sensors, which can acquire and provide information regarding the treatment area (e.g., a temperature, humidity, etc.), information regarding an individual within the treatment area (e.g., height, body temperature, presence or absence of contaminants, location of contaminants, etc.), and/or the like.

A visual and/or audible indicator can provide a user with information regarding an operating status of the treatment system, thereby enhancing user awareness and confidence in the treatment process. Such information can include an indication as to whether the far-UVC light is being emitted, an amount of time associated with a treatment (e.g., elapsed time, time remaining, etc.). Additionally, a visual and/or audible indicator can provide instructions for an individual receiving treatment, e.g., to change a location, change a position, etc.

A control unit can manage operation of the far-UVC light source(s) and corresponding sensing and information components to provide effective treatment and user safety.

In operation, an individual can trigger a sensor (e.g., a motion sensor) upon entering a treatment area, which can prompt activation of the far-UVC light source. As the individual passes through the treatment area, the far-UVC light directs light onto the individual and/or his/her clothing, which can effectively eradicate pathogens present on the individual's skin and/or clothing. Once all individuals have exited the treatment area, the sensor can detect the lack of any human presence and cause the control unit to deactivate the far-UVC light source.

Various embodiments can be implemented using an alternative solution. For example, any of various different configurations (e.g., amount, locations, power, types, etc.) of far-UVC light sources can be implemented to provide effective treatment for a given cost. Additionally, any of various sensor technologies, such as infrared or pressure sensors can be utilized for motion detection.

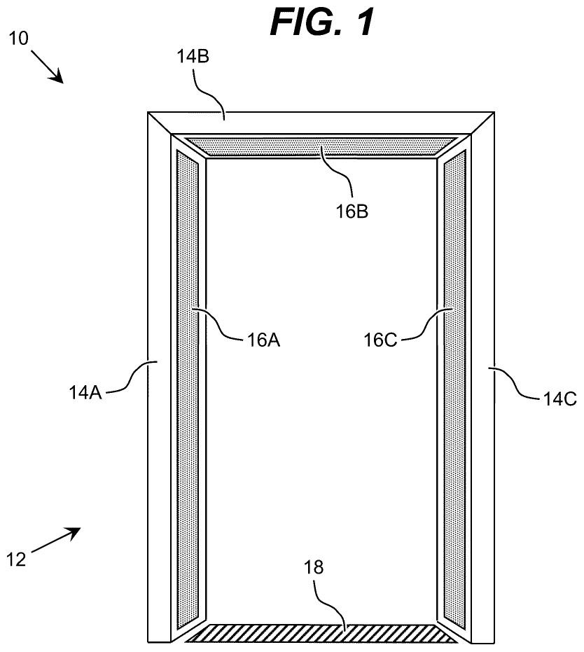

Turning to the drawings, FIG. 1 shows an illustrative ultraviolet treatment system 10 according to embodiments. The treatment system 10 includes a frame 12 constructed from panels 14A-14C. Each panel 14A-14C can be fabricated from any type of material, such as aluminum, etc. A panel 14A-14C can be sized to provide a treatment area located within a region defined by the panels 14A-14C of any desired size. In embodiments, the panels 14A-14C define a treatment area having an approximate height and width dimensions of a typical door. To this extent, in embodiments, the treatment area defined by the panels 14A-14C can have a height of approximately seven to eight feet and a width of approximately two to three and a half feet.

A depth of the panels 14A-14C and the corresponding treatment area can be configured based on an intended treatment time, treatment target, and/or the like. To this extent, in embodiments in which an individual merely walks through the treatment area, the depth can be relatively small, e.g., six inches to a foot. In embodiments in which an individual stands within the treatment area, the depth can be wider, e.g., one foot to three feet.

In embodiments, the frame 12 is configured to be readily relocated and set up in a desired location. To this extent, embodiments of the panels 14A-14C can be configured for ready disassembly and reassembly, e.g., through the use of transition fits (e.g., also referred to as a push fit or a snug fit) along connection surfaces.

Furthermore, each panel 14A-14C can enable expansion and/or reduction of the treatment area through modular expansion in any desired direction (e.g., height, width, or depth. To this extent, each panel 14A-14C can include transition fits that enable selective connection to another panel 14A-14C on any combination of the four edges of the panel 14A-14C.

As illustrated, a panel 14A-14C can have one or more edges having a forty-five degree angle to enable two panels to be secured and form a ninety degree angle.

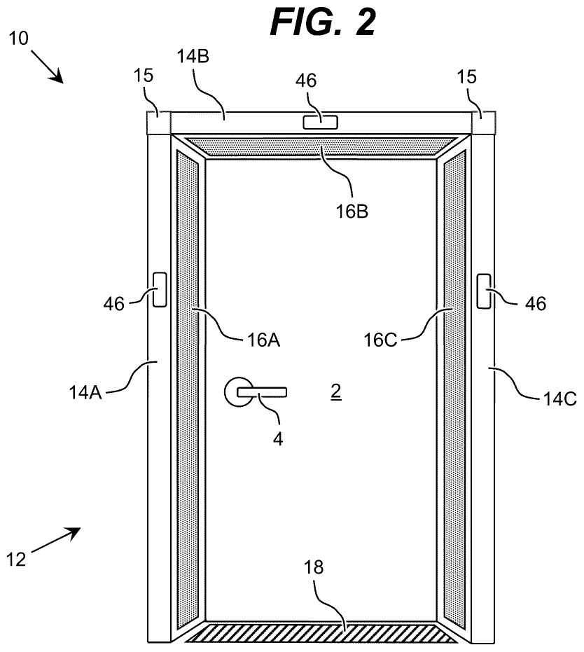

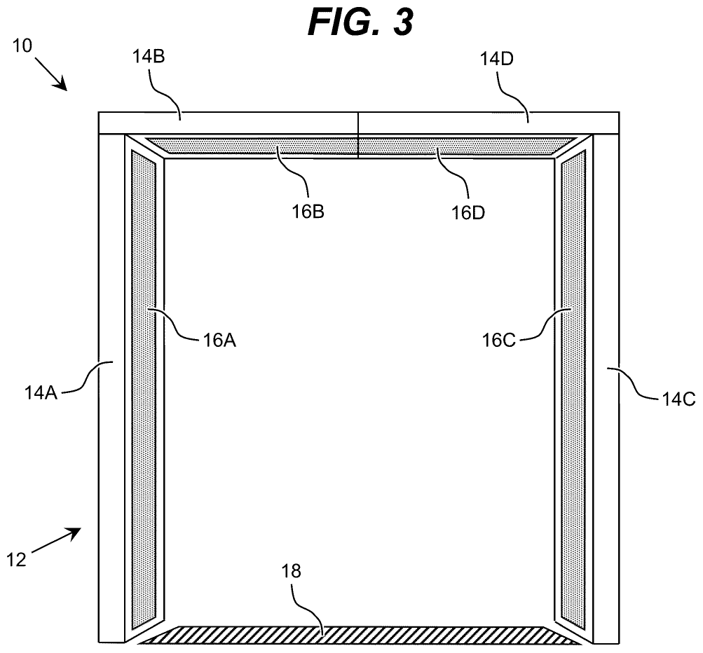

However, it is understood that this is only illustrative, and embodiments can include panels configured to be connected to a corner connector 15 (illustrated in FIG. 2), a rectangular edge along an outer perimeter of a facing side (illustrated in FIG. 3), abutting edges (illustrated in FIG. 3), and/or the like. Regardless, in each case a transition fit can provide a connection between the panels 14A-14C. In embodiments, a panel connection can be more permanently secured using any solution, e.g., with the use of one or more screws, nuts and bolts, and/or the like.

In embodiments, a panel 14A-14C can include a corresponding illumination component 16A-16C. Each illumination component 16A-16C can include one or more light sources configured to emit light directed into the treatment area. In embodiments, an illumination component 16A-16C can include an emission surface from which light generated by one or more light sources located within the panel 14A-14C is emitted into the treatment area. In embodiments, at least a portion of the emission surface is configured to emit diffuse light therefrom, e.g., through an emission surface that is roughened, frosted, and/or the like.

In embodiments, a panel 14A-14C can include a power and control interface at a corresponding connection to enable a corresponding illumination component 16A-16C to be operatively connected to a power source and/or a control unit. The power and control interface can comprise any type of interface, including a custom interface, which allows for the physical connection of wiring carrying power and/or data present in the two panels 14A-14C.

In embodiments, the treatment system 10 can include a sensor for detecting the presence of an individual within the treatment area. For example, embodiments can include a sensor mat 18, which can generate a signal when an individual is located on the sensor mat 18. In a more particular embodiment, the sensor mat 18 comprises a switch mat 18, which can generate a signal used to turn on or off the illumination component 16A-16C.

As described herein, embodiments of the treatment system 10 can be located adjacent to a door. To this extent, FIG. 2 shows an illustrative configuration of a treatment system 10 located in front of a door 2 according to embodiments. In embodiments, one or more panels 14A-14C can be configured to be attached to a wall, molding, and/or the like, located around the door 2 to prevent movement of the treatment system 10. In embodiments, a panel 14A, 14C can be configured to be secured to a location on a floor.

In embodiments, the treatment system 10 can interface with the door 2. For example, in embodiments, the door 2 can comprise an entry/exit for an area within which individuals must be decontaminated (e.g., a surgical room). In this case, when a door handle 4 is used from inside the room to allow an individual to exit the room, the treatment system 10 can keep the illumination components 16A-16C off, e.g., even as the individual is detected by a sensor, such as the sensor mat 18. However, when an individual seeks to enter the treatment area (e.g., as indicated by the sensor mat 18), the treatment system 10 can turn on the illumination components 16A-16C.

In embodiments in which the treatment must be performed for a minimum period of time, the treatment system 10 can prevent the individual from entering the area until the treatment has been completed. For example, the treatment system 10 can interface with a lock on the door handle 4 to prevent an individual from entering the area without first undergoing the minimum treatment. Once the treatment has completed, the treatment system 10 can unlock the door handle 4 and turn off the illumination components 16A, 16C. In further embodiments, a presence of an individual can be determined via the individual touching the door handle 4, which can include one or more sensors configured to generate a signal when the door handle 4 is touched by an individual.

Embodiments of a treatment system described herein can be implemented in various sizes. For example, FIG. 3 shows an illustrative treatment system 10 according to embodiments. In this case, the treatment system 10 is illustrated as being wider than a typical door. Such a configuration can be located at, for example, an entrance configured to accommodate a larger number of individuals entering or exiting an area, such as an arena, stadium, airport, or the like.

As illustrated, embodiments of the treatment system 10 can enable a wider configuration through the connection of two or more panels in alignment, such as panels 14B, 14D. While two aligned panels 14B, 14D are shown, it is understood that any number of panels can be connected in an aligned configuration to provide a treatment area having any desired width.

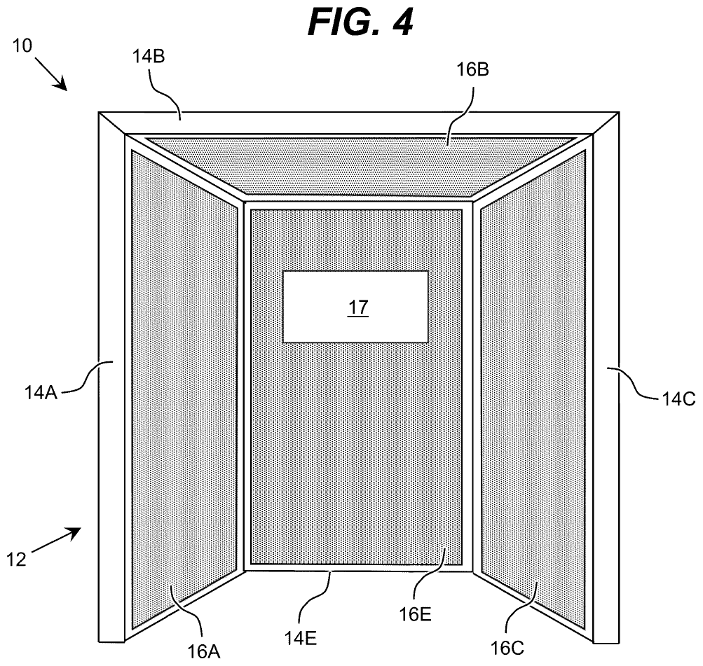

Embodiments of a treatment system described herein can comprise an at least partially enclosed walk-in booth within which treatment can be performed. For example, FIG. 4 shows an illustrative treatment system 10 according to embodiments. In these embodiments, the frame 12 is shown including panels 14A-14C, 14E that form a three-sided treatment area enclosed at the top. However, it is understood that this configuration is only illustrative. In other embodiments, the frame 12 can comprise a panel forming a floor of the treatment area, an open top, panels for each of the four sides, with one or more of the panels including a door, a panel that partially encloses the fourth side, and/or the like.

Regardless, each panel 14-14C, 14E can include a corresponding illumination component 16A-16C, 16E, respectively. Each illumination component 16A-16C, 16E can be configured to emit treatment light, e.g., far-UVC light, and/or another light, such as visible light. As illustrated, one or more of the illumination components can include an information display area 17. The information display area 17 can comprise an area within which sharper visible light-based light is emitted. In particular, the information display area 17 can comprise a liquid crystal display (LCD) or the like, which the control unit can operate to provide information to an individual located within the treatment booth. Such information can include an amount of time remaining in the treatment, a status of the treatment, whether a contaminant has been detected, etc. Additionally, the information display area 17 can be configured to enable the individual to make one or more selections regarding the treatment, halt a treatment, and/or the like. To this extent, the information display area 17 can comprise a touchscreen or the like, which can enable in individual to enter information processed by the control unit.

As discussed herein, embodiments of the panels 14A-14E described herein can be configured for repeated assembly and disassembly. To this extent, assembly of two panels can use transition fits, such as similar fits, which can provide a tight fit but allow for disassembly (with some force) and reassembly. In embodiments, a looser fit, such as a clearance fit, can be utilized and the panels 14A-14E can be secured using any of various approaches, such as a locking mechanism, e.g., set screw, pin, and/or the like.

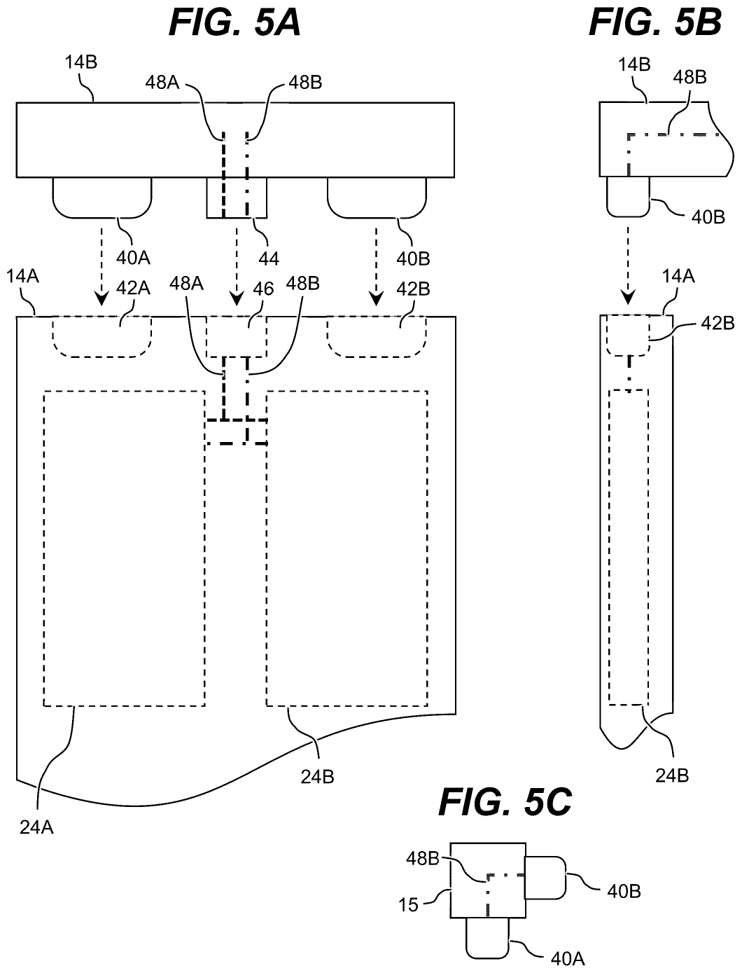

FIGS. 5A and 5B show schematic front and right side views, respectively, of an illustrative connection interface between two panels 14A, 14B according to embodiments. As illustrated, a first panel, such as panel 14B, can include a set of shafts 40A, 40B, which can be sized and located to securely fit into a corresponding set of openings 42A, 42B on a second panel, such as panel 14A. Additionally, the panel 14B can include a male connection interface 44 which is configured to be securely inserted into a female connection interface 46 located in the panel 14A. The connection interfaces 44, 46 can enable connection of power 48A and/or data 48B wiring between the panels 14A, 14B. As further shown with respect to the panel 14A, the power 48A and/or data 48B wiring can be connected to various components embedded in the panel 14A, such as far-UVC light sources 24A, 24B. In embodiments, both panels 14A, 14B can include openings 42A, 42B and removable shafts 40A, 40B can be inserted into the respective openings to secure the panels 14A, 14B.

In embodiments, the connection interface shown in FIGS. 5A and 5B can correspond to a connection interface between a top panel, such as panel 14B, and a bottom panel, such as panel 14A, shown in FIG. 3. However, it is understood that a similar connection interface can be utilized for each of the panel connections illustrated herein. To this extent, FIG. 5C shows a schematic of an illustrative corner connector 15 according to embodiments. As illustrated, a corner connector 15 can have a rectangular cross section and can include shafts 40A, 40B located on two adjacent sides. Additionally, while not shown, the corner connector 15 can include a male connection interface on the two adjacent sides which can enable connection of power and/or data 48B wiring between two panels connected to the respective sides. While not shown, it is understood that embodiments can include a straight connector in which shafts and/or openings and connection interfaces are located on opposing sides of the rectangular cross section.

In embodiments, a frame for a treatment system described herein can be fabricated using standardized and interchangeable panels, with or without connectors. For example, as illustrated in FIG. 2, each panel 14A, 14C can be a standardized long panel, while panel 14B can be a standardized short panel. The panels 14A-14C can be connected via standardized corner connectors 15 and/or straight connectors (not shown), which can enable any of various sizes and shapes for the treatment area formed there between. To this extent, in embodiments, each panel 14A-14C can include female connectors and openings on opposing ends, which can enable the panel 14A-14C to be secured in either of two orientations. In this case, adjacent panels can be connected via connectors, such as the corner connectors 15. While panels 14A-14C are shown and described as being configured for connections along their corresponding shorter sides, it is understood that embodiments of the panels 14A-14C can enable connection to other panels (directly or via a connector) along any combination of the four sides. To this extent, panels can be connected to form any of various desired heights, widths, and depths for the corresponding treatment area.

In embodiments, a panel 14A-14C also can include a connection interface which enables connection to an externally located control unit and/or power unit. For example, as illustrated in FIG. 2, each long side of the panels 14A-14C can include a female connection interface 46 to facilitate connection to a corresponding control unit and/or power unit. However, it is understood that the type, location, and number of connection interfaces 46 is only illustrative of various possible configurations.

It is understood that the types, combinations, and configurations of the various connectors, wiring, etc. shown in the drawings are only schematic representations of the components that illustrate the corresponding features. To this extent, embodiments are not limited to the particular types, combinations, and configurations of connectors, wiring, etc. shown in the drawings.

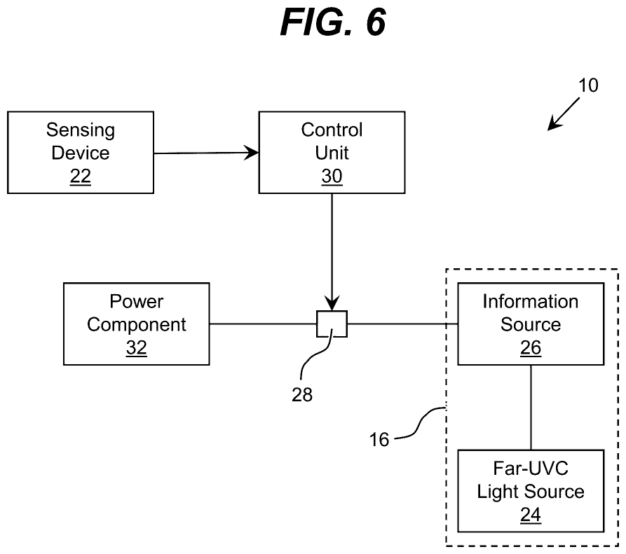

FIG. 6 shows an illustrative schematic diagram of operation of a treatment system 10 according to embodiments. As illustrated, a control unit 30 can receive data from one or more sensing devices 22. The data can include data corresponding to a presence or absence of an individual in a treatment area. Using the data, the control unit 30 can operate a switch 28, which selectively allows power to pass from a power component 32 to one or more illumination components 16. The control unit 30 can operate the switch 28 to cause the illumination component(s) 16 to emit far-UVC light such that a required treatment is performed, until no individual is present in the treatment area, a treatment time has expired, and/or the like.

In addition, the control unit 30 can use data acquired by one or more other types of sensing devices 22. Illustrative sensors can include a humidity sensor, which can detect a level of moisture within the treatment area, a visible light sensor, which can detect fluorescence from a contaminant located on a surface located within the treatment area, a chemical or gas sensor, which can detect amounts of one or more types of chemicals associated with a contaminant, and/or the like.

As illustrated, the illumination component 16 can include one or more information sources 26 and one or more far-UVC light source 24. In embodiments, the illumination component 16 can be configured such that the information source(s) 26 must be emitting light, such as visible light, for the far-UVC light source(s) 24 to be emitting far-UVC light. In this manner, an individual can be informed via the visible light emitted from the information source 26 when the far-UVC light is being emitted by the far-UVC light source 24. In embodiments, neither the information source 26 nor the far-UVC light source 24 emits light in the event of a failure of either light source.

However, it is understood that this is only an illustrative configuration of an illumination component 16. In embodiments, an illumination component 16 can include one or more sensors, which can sense far-UVC light and send an indication to the control unit 30 as to whether far-UVC light is being emitted or not.

In embodiments, the treatment is configured to disinfect various surfaces associated with a human, including the skin and clothing being worn by the individual.

For suitable disinfection, a dose of 0.5 to 1.0 millijoules per square centimeter (mJ/cm²) is generally utilized, with radiant flux values ranging from 0.1 to 2.0 milliwatts per square centimeter (mW/cm²). A duration of the treatment (e.g., exposure to the far-UVC) needed to achieve such a dose depends on the irradiance level. For instance, at 0.5 mW/cm², a duration of 1 to 2 seconds is usually sufficient to deliver the above-referenced dose. These parameters can ensure both effective pathogen neutralization and safety in practical applications.

In embodiments, when the treatment area is sufficiently long for an individual to take at least one or two seconds to traverse therethrough at a fast paced walk, the treatment area can be located such that the individual does not need to stop during treatment. However, in embodiments in which a thorough treatment is required while ensuring all surfaces are treated, an individual may be required to stop and perform one or more actions (e.g., arm and/or leg movements, change in facing direction, etc.) to ensure a thorough treatment.

Regardless, embodiments can turn off the far-UVC lights in response to detecting that an individual has remained present within the treatment area for a period of time exceeding a maximum period of time. In embodiments, the maximum period of time is approximately 3-5 times a maximum required duration for the treatment. To this extent, for a treatment period of 1-2 seconds, the maximum period of time can be set to be between 3-10 seconds. In embodiments, the information source 26 can provide an indication that the treatment has stopped. For example, a visible light can change a different color, a visible light can be turned off, a message can be displayed/audibled, etc.

In embodiments, the information source 26 can comprise a visible light source that emits diffuse light when the far-UVC light source 24 is emitting far-UVC light. In these embodiments, emission/non-emission of the visible light informs the individual of a status of the treatment. In embodiments, the information source 26 can comprise a display that presents text, graphics, or other visual indicia regarding the status of the treatment. In still further embodiments, the information source 26 can comprise an audio device, which emits sounds, audible commands, audible information, and/or the like, associated with the treatment.

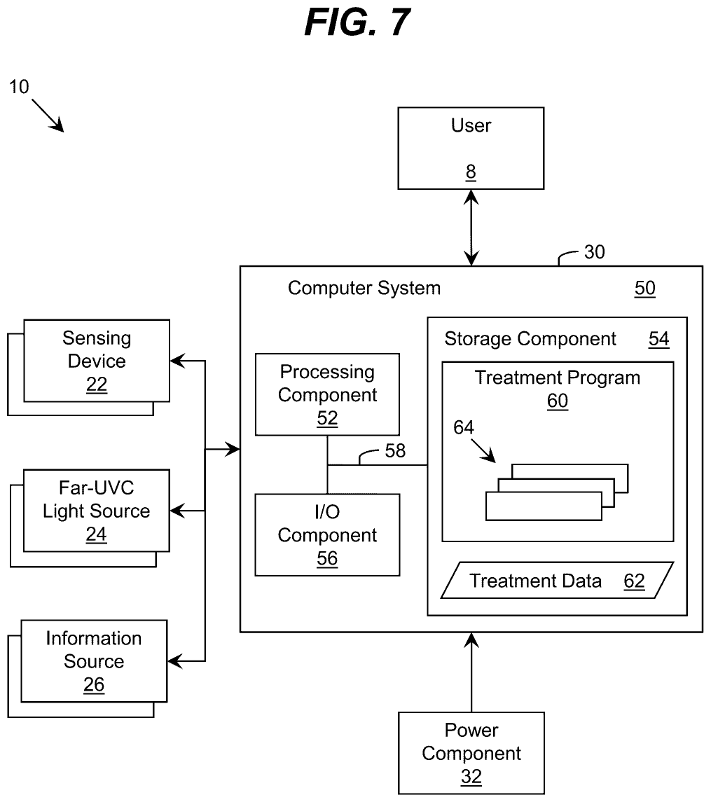

FIG. 7 shows a schematic of an illustrative treatment system according to embodiments. To this extent, the treatment system 10 includes a control unit 30, which is implemented as a computer system 50 that can perform a process described herein in order to perform a far-UVC treatment. In particular, the computer system 50 is shown including a treatment program 60, which makes the computer system 50 operable to treat an area by performing a process described herein.

The computer system 50 is schematically illustrated as including a processing component 52 (e.g., one or more processors), a storage component 54 (e.g., a storage hierarchy), an input/output (I/O) component 56 (e.g., one or more I/O interfaces and/or devices), and a communications pathway 58. In general, the processing component 52 executes program code, such as the treatment program 60, which is at least partially fixed in storage component 54. While executing program code, the processing component 52 can process data, which can result in reading and/or writing transformed data from/to the storage component 54 and/or the I/O component 56 for further processing. The pathway 58 provides a communications link between each of the components in the computer system 50.

The I/O component 56 can comprise one or more human I/O devices, which enable a human user 8 to interact with the computer system 50 and/or one or more communications devices to enable a system user 8 (e.g., a portable computing device of a human user, such as a mobile phone executing an app) to communicate with the computer system 50 using any type of communications link. To this extent, the treatment program 60 can manage a set of interfaces (e.g., graphical user interface(s), application program interface, and/or the like) that enable human and/or system users 8 to interact with the treatment program 60 and the treatment data 62, etc. Furthermore, the treatment program 60 can manage (e.g., store, retrieve, create, manipulate, organize, present, etc.) the treatment data 62 using any solution.

In any event, the computer system 50 can comprise one or more general purpose computing articles of manufacture (e.g., computing devices) capable of executing program code, such as the treatment program 60, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the treatment program 60 can be embodied as any combination of system software and/or application software.

Furthermore, the treatment program 60 can be implemented using a set of modules 64. In this case, a module 64 can enable the computer system 50 to perform a set of tasks used by the treatment program 60, and can be separately developed and/or implemented apart from other portions of the treatment program 60. As used herein, the term “component” means any configuration of hardware, with or without software, which implements the functionality described in conjunction therewith using any solution, while the term “module” means program code that enables a computer system 50 to implement the actions described in conjunction therewith using any solution. Regardless, it is understood that two or more components, modules, and/or systems may share some/all of their respective hardware and/or software. Furthermore, it is understood that some of the functionality discussed herein may not be implemented or additional functionality may be included as part of the computer system 50.

When the computer system 50 comprises multiple computing devices, each computing device can have only a portion of the treatment program 60 fixed thereon (e.g., one or more modules 64). In embodiments, the computer system 50 can comprise a computing unit located within a panel and a portable computing unit, such as a mobile phone, which is executing an app installed thereon for enabling a user 8 to monitor, evaluate, manage, and/or the like, the treatment system 10.

However, it is understood that the computer system 50 and the treatment program 60 are only representative of various possible equivalent computer systems that may perform a process described herein. To this extent, in other embodiments, the functionality provided by the computer system 50 and the treatment program 60 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 50 includes multiple computing devices, the computing devices can communicate over any type of communications link. Furthermore, while performing a process described herein, the computer system 50 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

Embodiments described herein can include features described in conjunction with a system capable of detecting and/or sterilizing surface(s) of an object as shown and described in U.S. Patent Application Publication No. 2016/0106873, which is hereby incorporated by reference.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength +/−five nanometers) by an active region of an optoelectronic device during operation of the device. For a given layer, the wavelength can be measured in a material of consideration and can depend on a refractive index of the material.

It is understood that, unless otherwise specified, each value is approximate and each range of values included herein is inclusive of the end values defining the range. Terms of degree such as “generally,” “substantially,” “about,” and “approximately” as used herein mean a reasonable amount of deviation of the modified term such that the end result is not significantly changed. For example, these terms can be construed as including a deviation of at least +/−0.5% of the modified term if this deviation would not negate the meaning of the word it modifies. In a more particular example, the term “approximately” is inclusive of values within +/− ten percent of the stated value, while the term “substantially” is inclusive of values within +/− five percent of the stated value when these deviations would not negate the meaning of the word each term modifies. Unless otherwise stated, two values are “similar” when the amount of deviation between the two values does not significantly change the result. In a more particular example, two values are similar when the smaller value is within +/− twenty-five percent of the larger value. A value, y, is on the order of a stated value, x, when the value y satisfies the formula 0.1x≤y≤10x.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A surface treatment system comprising:
a reconfigurable frame structure defining at least two sides of a treatment area located between the at least two sides, wherein the frame structure comprises a plurality of panels configured for repeated assembly and disassembly in any of a plurality of configurations;
a plurality of far-UVC light sources located in the frame structure, wherein the plurality of far-UVC light sources are configured to emit far-UVC light directed into the treatment area;
a set of motion sensors configured to acquire presence data corresponding to a presence of an object in the treatment area; and
a control unit configured to operate the plurality of far-UVC light sources to treat surfaces of the object located in the treatment area using the presence data.

2. The system of claim 1, wherein the far-UVC light has at least one peak wavelength, wherein each of the at least one peak wavelength is between 207 nanometers and 222 nanometers.

3. The system of claim 1, wherein the object comprises a human.

4. The system of claim 3, wherein the treatment area corresponds to an entrance through which individuals pass to enter or exit a defined area.

5. The system of claim 1, wherein the frame structure further defines a top of the treatment area, and wherein at least one of the plurality of far-UVC light sources is located in the top of the frame structure.

6. The system of claim 1, wherein the frame structure further defines a bottom of the treatment area, and wherein at least one of the set of motion sensors is located in the bottom of the frame structure.

7. The system of claim 6, wherein the bottom further includes at least one of the plurality of far-UVC light sources.

8. The system of claim 1, wherein the control unit is configured to end treatment of the object located within the treatment area in response to a duration for which the object has been located in the treatment area exceeding a maximum treatment duration.

9. The system of claim 1, wherein the frame structure defines a total depth of the treatment area of no more than approximately one foot.

10. The system of claim 1, wherein the panels are assembled using a plurality of transition fits.

11. The system of claim 1, further comprising at least one sensor configured to acquire approach data for an object approaching the treatment area from at least one side of the treatment area, wherein the control unit is configured to selectively treat the surfaces of the object using the approach data.

12. A treatment system comprising:
a frame structure comprising a plurality of panels defining a top and two sides of a treatment area, wherein each of the plurality of panels includes at least one far-UVC light source configured to emit far-UVC light directed into the treatment area;
a set of sensors configured to acquire presence data corresponding to a presence of an object in the treatment area; and
a control unit configured to operate the plurality of far-UVC light sources to treat surfaces of the object using the presence data.

13. The system of claim 12, wherein the panels are secured to each other using a set of transition fits.

14. The system of claim 12, wherein a connection between two adjacent panels includes a power and control connection interface.

15. The system of claim 12, further comprising at least one sensor configured to acquire approach data corresponding to an object approaching the treatment area from at least one side of the treatment area, wherein the control unit is further configured to operate the plurality of far-UVC light sources using the approach data.

16. The system of claim 15, wherein the control unit is configured to use the approach data to selectively treat the surfaces of the object based on a side from which the object enters the treatment area.

17. The system of claim 12, wherein the frame structure defines a walk-in booth for the treatment area.

18. A method of treating surfaces associated with a human, the method comprising:
assembling a reconfigurable frame structure including a plurality of panels configured for repeated assembly and disassembly into one of a plurality of configurations, wherein the assembled reconfigurable frame structure at least partially defines a treatment area, and wherein each of the plurality of panels includes at least one far-UVC light source configured to emit far-UVC light directed into the treatment area;
acquiring presence data corresponding to a presence of the human in the treatment area using a set of motion sensors; and
a control unit operating the plurality of far-UVC light sources to treat the surfaces associated with the human located in the treatment area using the presence data, wherein the surfaces include human skin and clothing being worn by a human.

19. The method of claim 18, further comprising:
the control unit receiving approach data acquired by at least one sensor for a human approaching the treatment area from at least one side of the treatment area; and
the control unit selectively operating the plurality of far-UVC light sources to treat the surfaces using the approach data.

20. The method of claim 18, wherein the control unit operating includes ending treatment of the surfaces in response to a duration for which the human has been located in the treatment area exceeding a maximum treatment duration.
